(19) United States
(12) Patent Application Publication
(10) Pub. No.: US 2026/0047894 A1
(43) Pub. Date: Feb. 19, 2026

(54) System and Method for Tracking and Steering Medical Devices

(71) Applicant: Bard Access Systems, Inc., Salt Lake City, UT (US)

(72) Inventors: Zachary Jarrod Traina, Denville, NJ (US); Shayne Messerly, Kaysville, UT (US)

(21) Appl. No.: 18/808,975

(22) Filed: Aug. 19, 2024

(51) Int. Cl. A61B 34/20; A61B 5/06; A61B 90/00

(52) U.S. Cl. CPC A61B 34/20; A61B 5/062; A61B 90/37; A61B 2034/2061

(57) ABSTRACT

A tracking and steering system for medical devices comprises a magnetic element attached to a distal portion of the medical device, a sensor on the patient's external surface detecting the magnetic field strength of the medical device's magnetic element, and a steering magnet on the patient's external surface with an electromagnet attracting the medical device's magnetic element. A console with memory and processors receives sensor data to determine the medical device's distal tip position and controls the sensor and steering magnet activation. A display shows an image of the patient with an icon indicating the medical device's location relative to the patient, enhancing medical procedures with real-time tracking and steering capabilities.

### BACKGROUND

Advancing medical devices through tortuous vascular pathways can be challenging especially when directing the medical device distal tip at vascular junctions. External magnets can be used to modify a position of the medical device distal tip either by attracting, repelling or urging the distal tip through various vascular pathways. However, since the medical device distal tip cannot be directly observed, positioning the external steering magnet relative to the medical device distal tip can be challenging.

Various tracking systems can be used to determine positional information of the medical device distal tip within the vasculature. However, the clinician often needs to divide their attention between the imaging system display and the steering magnet disposed on the external surface of the patient. Moreover, depending on the modality of the tracking system employed, the steering magnet can also interfere with the functionality of the tracking system or other medical devices, such as pacemakers, disposed within the patient. What is needed, therefore, is a system and method to address the foregoing.

### SUMMARY

In some aspects, the techniques described herein relate to a tracking and steering system including, a medical device including a magnetic element coupled with a distal portion of the medical device, a sensor placed on an external surface of a patient and configured to detect a magnetic field strength of the magnetic element of the medical device, a steering magnet disposed on the external surface of the patient and including an electromagnet configured to attract the magnetic element of the medical device, a console including memory and one or more processors configured to receive magnetic field strength information from the sensor and determine positional information for a distal tip of the medical device, and simultaneously activate the sensor and deactivate the steering magnet or simultaneously activate the steering magnet and deactivate the sensor, and a display configured to receive positional information from the console and display an image of the patient and display an icon representing a location of the medical device relative to the patient.

In some aspects, the techniques described herein relate to a tracking and steering system, wherein the medical device includes one or more of a catheter, guidewire, or stylet.

In some aspects, the techniques described herein relate to a tracking and steering system, wherein the magnetic element of the medical device is a permanent magnet or a magnetically responsive material.

In some aspects, the techniques described herein relate to a tracking and steering system, wherein the console is configured to receive an input from a clinician to switch between simultaneously activating the sensor and deactivating the steering magnet, and simultaneously activating the steering magnet and deactivating the steering magnet.

In some aspects, the techniques described herein relate to a tracking and steering system, wherein the console is further configured to switch between simultaneously activating the sensor and deactivating the steering magnet, and simultaneously activating the steering magnet and deactivating the steering magnet at between 5 Hz and 50 Hz.

In some aspects, the techniques described herein relate to a tracking and steering system, wherein the positional information includes location, orientation, or shape of the medical device.

In some aspects, the techniques described herein relate to a tracking and steering system, wherein the steering magnet is formed integrally with the sensor to align the steering magnet with a predefined location when the sensor is disposed over a sternum of the patient.

In some aspects, the techniques described herein relate to a tracking and steering system, wherein the steering magnet is formed as a separate standalone device from the sensor and is communicatively coupled with one or both of the sensor and the console by wired or wireless communication.

In some aspects, the techniques described herein relate to a tracking and steering system, wherein the steering magnet coupled to a garment that is worn by the patient and aligns the steering magnet with a predetermined location on an exterior of the patient.

In some aspects, the techniques described herein relate to a tracking and steering system, wherein one or both of the sensor and the medical device includes an electrode configured to detect an electrical impulse, and wherein the console is configured to determine a presence of a pacemaker, and in response to the console determining the presence of the pacemaker, the console prevents the steering magnet from being activated.

In some aspects, the techniques described herein relate to a tracking and steering system, wherein the console provides one or more of a visual, audible, and tactile alert to a clinician to indicate the steering magnet is deactivated due to the presence of the pacemaker being detected.

In some aspects, the techniques described herein relate to a tracking and steering system, wherein a polarity of the electromagnet is reversed to change the steering magnet between attracting and repelling the medical device magnetic element.

In some aspects, the techniques described herein relate to a tracking and steering system including, a medical device including, a first magnetic element coupled with a distal portion thereof, and a first integrated optical-fiber having a plurality of fiber Bragg grating (“FBG”) sensors along a portion thereof, a steering magnet disposed on an external surface of a patient including, a second magnetic element, and a tether extending from the steering magnet and including a second integrated optical-fiber having a plurality of FBG sensors along a portion thereof, a console including memory and one or more processors configured to, convert FBG sensor-reflected optical signals from the first integrated optical-fiber in the medical device and the second integrated optical-fiber in the tether into plottable data by way of a plurality of optical signal-converter algorithms to determine position information for a distal tip of the medical device and the steering magnet, and a display configured to provide an image of the patient, a first icon indicating a position of the medical device, and a second icon indicating a position of the steering magnet relative to each other.

In some aspects, the techniques described herein relate to a tracking and steering system, wherein the medical device includes one of a catheter, guidewire, or stylet.

In some aspects, the techniques described herein relate to a tracking and steering system, wherein the first magnetic element of the medical device is one of a permanent magnet or a magnetically responsive material, and wherein the second magnetic element of the steering magnet is one of a permanent magnet or an electromagnet.

In some aspects, the techniques described herein relate to a tracking and steering system, wherein a positional information of the distal tip of the medical device and the steering magnet includes location, orientation, or shape.

In some aspects, the techniques described herein relate to a tracking and steering system, wherein the tether is coupled to the console.

In some aspects, the techniques described herein relate to a tracking and steering system, wherein the console further includes one or more logic engines configured to determine a position of the distal portion of the medical device relative to a position of the steering magnet and activate the steering magnet when the distal portion of the medical device approaches a target location and deactivate the steering magnet when the distal portion of the medical device is advanced past the target location.

In some aspects, the techniques described herein relate to a tracking and steering system including, a medical device including a magnetic element coupled with a distal portion of the medical device, a sensor placed on an external surface of a patient and configured to detect a magnetic field strength of the magnetic element of the medical device, a steering magnet disposed on the external surface of the patient and including an electromagnet configured to attract the magnetic element of the medical device, the steering magnet including a tether having an integrated optical-fiber having a plurality of fiber Bragg grating (“FBG”) sensors along a portion thereof, a console including memory and one or more processors configured to, receive magnetic field strength information from the sensor and determine positional information for a distal tip of the medical device, simultaneously activate the sensor and deactivate the steering magnet or simultaneously activate the steering magnet and deactivate the steering magnet, and convert FBG sensor-reflected optical signals from the integrated optical-fiber in the tether into plottable data by way of a plurality of optical signal-converter algorithms to determine position information for the steering magnet, and a display configured to receive positional information from the console and display an image of the patient, display a first icon representing a location of the medical device relative to the patient, and a second icon representing a location of the steering magnet.

In some aspects, the techniques described herein relate to a method of placing a medical device including, advancing a distal tip of the medical device intravascularly, the distal tip including a medical device magnetic element, placing a steering magnet on an external surface of a patient to modify a position of the distal tip disposed intravascularly, the steering magnet including an electromagnetic element, detecting a magnetic field strength of the medical device magnetic element by way of a sensor placed on the external surface of the patient, and modifying an activation state of the steering magnet and the sensor, by way of a console, to alternate between simultaneously activating the steering magnet and deactivating the sensor, and simultaneously deactivating the steering magnet and activating the sensor at a rate of between 5 Hz and 50 Hz.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIGS. 1A-1C show a perspective view of a steering magnet modifying a position of a medical device distal tip disposed intravascularly, in accordance with embodiments disclosed herein.

FIG. 2 shows a perspective view of a garment worn by a patient for securing one or more steering magnets to an external surface of a patient, in accordance with embodiments disclosed herein.

FIG. 3 shows a perspective view of a fiber optic enabled tracking and steering system for tracking and modifying a position of a medical device distal tip disposed intravascularly, in accordance with embodiments disclosed herein.

FIG. 4 shows a perspective view of a magnetic tracking and steering system for tracking and modifying a position of a medical device distal tip disposed intravascularly, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein. It is understood that the drawings are diagrammatic and schematic representations of exemplary embodiments of the invention and are neither limiting nor necessarily drawn to scale.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “right,” “left,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. Also, the words “including,” “has,” and “having,” as used herein, including the claims, shall have the same meaning as the word “comprising.”

In the following description, the terms “or” and “and/or” as used herein are to be interpreted as inclusive or meaning any one or any combination. As an example, “A, B or C” or “A, B and/or C” mean “any of the following, A, B, C, A and B, A and C, B and C, A, B and C.” An exception to this definition will occur only when a combination of elements, components, functions, steps or acts are in some way inherently mutually exclusive.

With respect to “proximal,” a “proximal portion” or a “proximal end portion” of, for example, a catheter or system disclosed herein includes a portion of the catheter or system intended to be near or relatively nearer to a clinician when the catheter or system is used on a patient. Likewise, a “proximal length” of, for example, the catheter or system includes a length of the catheter or system intended to be near or relatively nearer to the clinician when the catheter or system is used on the patient. A “proximal end” of, for example, the catheter or system includes an end of the catheter or system intended to be near or relatively nearer to the clinician when the catheter or system is used on the patient. The proximal portion, the proximal end portion, or the proximal length of the catheter or system can include the proximal end of the catheter or system; however, the proximal portion, the proximal end portion, or the proximal length of the catheter or system need not include the proximal end of the catheter or system. That is, unless context suggests otherwise, the proximal portion, the proximal end portion, or the proximal length of the catheter or system is not necessarily a terminal portion or terminal length of the catheter or system.

With respect to “distal,” a “distal portion” or a “distal end portion” of, for example, a catheter or system disclosed herein includes a portion of the catheter or system intended to be near or relatively nearer to a patient when the catheter or system is used on a patient. Likewise, a “distal length” of, for example, the catheter or system includes a length of the catheter or system intended to be near or relatively nearer to the patient when the catheter or system is used on the patient. A “distal end” of, for example, the catheter or system includes an end of the catheter or system intended to be near or relatively nearer to the patient when the catheter or system is used on the patient. The distal portion, the distal end portion, or the distal length of the catheter or system can include the distal end of the catheter or system; however, the distal portion, the distal end portion, or the distal length of the catheter or system need not include the distal end of the catheter or system. That is, unless context suggests otherwise, the distal portion, the distal end portion, or the distal length of the catheter or system is not necessarily a terminal portion or terminal length of the catheter or system.

As used herein, the term “location” is used to indicate a location of the medical device, or portion thereof, in three-dimensional space. By way of example, a distal tip of a medical device can be located at a location defined by three-dimensional co-ordinate. As used herein, the term “orientation” is used to indicate an orientation of the medical device at its location. By way of example, a longitudinal axis of a distal tip of the medical device can be oriented to be aligned along a particular direction in three-dimensional space at a specific location. As used herein, the term “shape” is used to indicate a plain shape of the medical device. By way of example, a ‘J’ shape of a medical device can include an elongate medical device with a curved tip. As used herein, the term “position” combines one or more aspects of the location, orientation and/or shape of a medical device. By way of example, at least a distal portion of the medical device can be in malpositioned when the distal portion of the medical device is folded over itself such that a distal tip of the medical device is oriented away from a desired orientation.

Unless defined otherwise, all technical and scientific terms used herein have the same meaning as commonly understood by those of ordinary skill in the art.

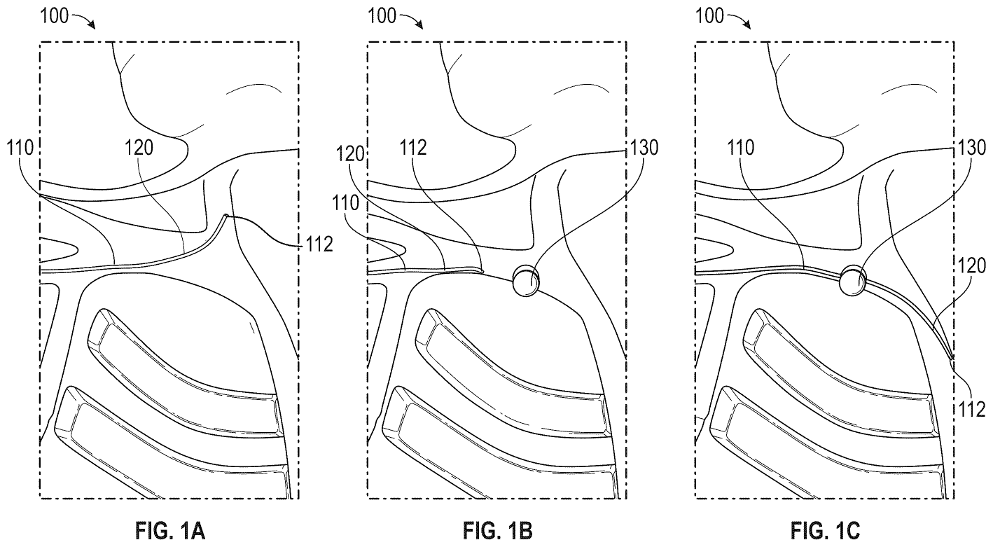

FIGS. 1A-1C show an embodiment of a medical device steering system (“system”) 100 generally including a medical device 110 including a magnetic element 120 and configured to be disposed within a vasculature, and a steering magnetic element 130 disposed external to the body of the patient 90, such as on a skin surface. Exemplary medical devices 110 include catheters, peripherally inserted central catheters (PICC), rapidly insertable central catheters (RICC), central venous catheters (CVC), midline catheters, intravenous (IV) catheters, guidewires, stylets, trocars, combinations thereof, or the like.

As shown in FIG. 1A, when advancing a medical device 110 through tortuous vascular pathways, the distal tip 112 of the medical device 110 can often become snagged at vascular junctions, preventing further distal movement or advancing down incorrect vascular pathways. To facilitate the steering the medical device distal tip 112 through the tortuous vascular pathways, the medical device 110 further includes one or more magnetic elements 120 disposed along a longitudinal length of the medical device 110. For example, the one or more magnetic elements 120 can be disposed at a distal tip 112, along a portion of the medical device 110, or along an entire length of the medical device 110, or combinations thereof. As used herein the magnetic element 120 of the medical device 110 can include a permanent magnet (e.g., neodymium magnet, or the like), an electro-magnet, or a magnetically receptive material (e.g., ferrous metals, or the like). In an embodiment, the system 100 further includes a steering magnet 130 disposed externally. The steering magnet 130 can include a permanent magnet (e.g., neodymium magnet, or the like), an electro-magnet, or a magnetically receptive material (e.g., ferrous metal, or the like).

As shown in FIG. 1B, the steering magnet 130 can be placed externally to the patient 90 and adjacent vascular junction to redirect the distal tip 112 of the medical device 110 towards the desired vascular pathway, as shown in FIG. 1C. In an embodiment, the steering magnet 130 and the medical device magnetic element 120 can be arranged with unlike-poles adjacent so that the steering magnet 130 attracts the medical device magnetic element 120, and portion of the medical device 110 the magnetic element 120 is coupled to, towards each other. In an embodiment, the steering magnet 130 can remain stationary at a predetermined external location to draw the distal tip 112 towards the desired vascular pathway as the distal tip 112 is advanced past the vascular junction adjacent the steering magnet 130. In an embodiment, the steering magnet 130 can be positioned adjacent a desired vascular pathway and attract the medical device magnetic element 120 with sufficient magnetic force so that moving the steering magnet 130 along the desired vascular pathway draws the medical device distal tip 112 through the vascular pathway.

In an embodiment, the steering magnet 130 and the medical device magnetic element 120 can be arranged with like-poles adjacent so that the steering magnet 130 repels the magnetic element 120, and portion of the medical device 110 the magnetic element 120 is coupled to, away from each other. In an embodiment, one or more steering magnets 130 with like poles can be positioned at one or more junctions along the desired vascular pathway, so as to repel the medical device distal tip 112 away from undesired vascular junctions and towards the desired vascular pathway.

In an embodiment, the steering magnet 130 includes an electromagnet. The poles of the steering magnet 130 can be switched depending on the direction of the current and as such, the steering magnet 130 can be switched between attracting the magnetic element 120 of the medical device 110 and repelling the magnetic element 120. In an embodiment, the system 100 can switch polarity of the steering magnet 130 in response to an input received from the clinician. Further the strength of the electromagnet can be modified by a change in current of the electromagnet in response to an input from the clinician.

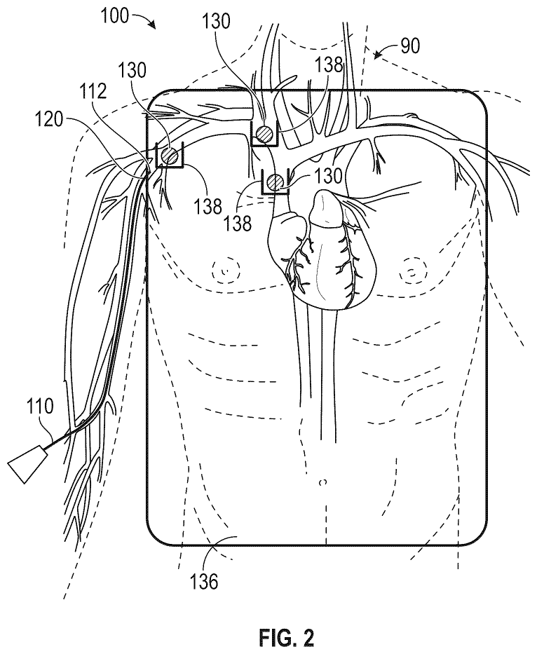

As shown in FIG. 2, in an embodiment, the one or more steering magnets 130 can be secured to a garment 136, such as a drape, vest, gown, T-shirt, apron, or the like. The garment 136 can be worn by the patient 90, draped over the patient 90, and/or aligned with one or more fiduciary points on the patient 90 to align a steering magnet 130 with a predetermined position on the patient 90. In an embodiment, the one or more steering magnets 130 can be sewn into the garment 136 at one or more predetermined positions. In an embodiment, the garment 136 includes one or more pockets 138 disposed at one or more positions over the surface of the garment 136. Each pocket of the one or more pockets 138 can be configured to receive and retain one or steering magnets 130. As such, a clinician can selectively place a steering magnet 130 at one or more positions over the surface of the garment 136, depending if a steering magnet 130 is needed at a position or not.

In an embodiment, the one or more pockets 138 can be placed regularly over the surface of the garment 136. In an embodiment, the one or more pockets 138 can be placed at one or more predetermined or strategic positions over the garment 136. In an embodiment, the surface of the garment 136 can include an image or an illustration of one or more anatomical features of the patient. Such anatomical features can include internal, external, target locations, fiduciary locations, such as heart, lungs, navel, pectoral muscles, major blood vessels, combinations thereof, or the like. As such, the illustrations can facilitate aligning the garment 136 with the anatomy of the patient 90 disposed below and facilitate illustrating to the clinician where to place the one or more steering magnets 130 and where the position relates to the anatomy of the patient 90. The garment 136 provides an intuitive and easy to use way of positioning steering magnets 130 on the body of the patient 90.

In an embodiment, the surface of the garment 136 can include hook and loop, or similar releasable fastening system configured to allow a clinician to releasably secure the one or more steering magnets 130 to surface of the garment 136. For example, the steering magnet 130 can include the “hook” material and the surface of the garment 136 can include the “loop” material, although other configurations are also contemplated. The “loop” material on the garment 136 can be disposed over the entire surface of the garment 136. Alternatively, the “loop” material can be placed on one or more predetermined positions over the surface of the garment 136. Other exemplary fastening systems include low tack adhesives, snap-fit engagements, snap fasteners, buttons, toggles, or the like.

### Medical Imaging

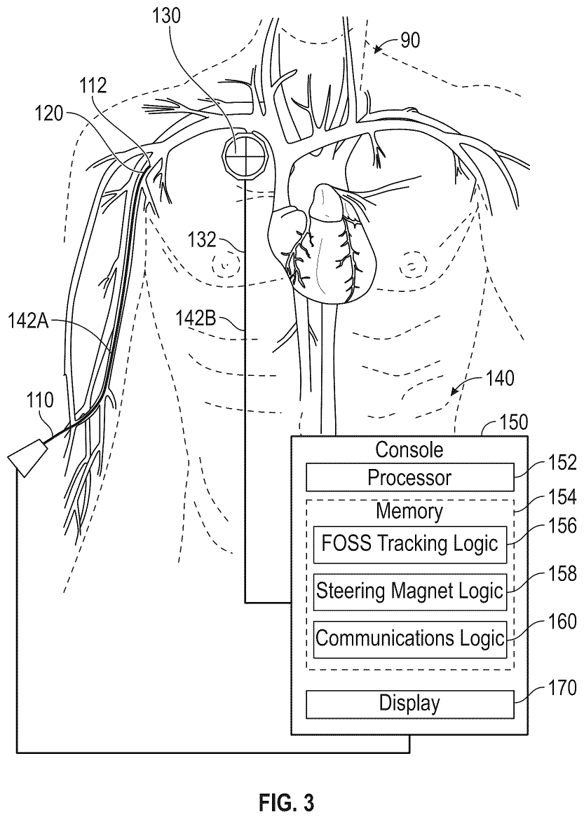

As will be appreciated, when a distal portion of the medical device 110 is disposed within the vasculature, the distal tip 112 cannot be directly observed by a clinician and as such, the system 100 can further include one or more medical imaging systems to detect and track the positional information, such as location, orientation, and shape of the medical device, or portion thereof, within the body of the patient 90. As shown in FIG. 3, in an embodiment, the system 100 further includes a medical imaging system 140 having a fiber-optic shape sensing (“FOSS”) modality to determine positional information of the medical device 110 within the vasculature.

In an embodiment, the FOSS medical imaging system 140 includes a medical device 110 having a first integrated optical-fiber 142A formed therewith. The integrated optical-fiber can be disposed within a wall of the medical device 110, within a lumen of the medical device 110, in a guidewire or stylet disposed within a lumen of the medical device 110, combinations thereof, or the like.

The integrated optical-fiber includes a one or more fiber Bragg grating (“FBG”) sensors along at least a portion of the optical-fiber configured to detect a shape of the optical-fiber and the associated medical device 110 in three-dimensional space. A proximal end of the medical device 110 and optical-fiber assembly is coupled with a console 150. The console 150 includes an interrogation light source that is provided to the optical-fiber. The interrogation light source impinges on the FBG sensors and provides a reflected, signal light. The reflected signal light indicates an angle and direction of deviation of a portion of the medical device 110 from a central longitudinal axis. The console 150 includes one or more processors 152, non-transitory memory or data storage 154, and one or more logic engines, for example a FOSS tracking logic 156, a steering magnet logic 158, and a communications logic 160. The FOSS tracking logic 156 can receive information from the one or more FBG sensors to determine an angle and direction of one or more portions of the medical device 110 and determine a shape of the medical device 110 in three-dimensional space.

The console 150 can then map the positional information of the medical device determined by the FOSS logic 156 against one or more images of the patient anatomy. Exemplary images of the patient anatomy can include generic images of a patient anatomy having one or more anatomical landmarks, or can include images specific to the individual patient, as well as ultrasound images, PET/CT images, MRI images, fluoroscopic images, combinations thereof or the like. These images can be provided either in real-time or can be captured previously and used to recreate a two dimensional or three-dimensional image of the patient anatomy. The console 150 can further include a display 170 to display an image of the patient anatomy and the medical device 110 mapped onto the patient anatomy. In an embodiment, the console 150 further includes a projector to project an image of the patient anatomy and/or medical device 110 onto the patient skin surface. Advantageously, this allows for clinician to observe only a single location rather than dividing their attention between the patient in a first location and a display in a second location. In an embodiment, the console 150 further includes a virtual reality and/or augmented reality projector.

Further details, examples and embodiments of FOSS enabled tracking systems can be found, for example, in the following U.S. patents and published patent applications: U.S. Ser. Nos. 11,474,310, 11,931,179, 11,899,249, 11,931,112, 11,850,338, 11,525,670, 11,622,816, 11,883,609, 11,624,677, 11,630,009, U.S. 2021/268229, U.S. 2021/275256, U.S. 2022/152349, U.S. 2022/160209, U.S. 2023/285085, U.S. 2023/346314, U.S. 2023/293243, U.S. 2023/346482, U.S. 2023/292997, U.S. 2023/320663, U.S. 2022/330891, U.S. 2023/338090, U.S. 2023/417998, U.S. 2024/016425, U.S. 2023/082991, U.S. 2024/180470, U.S. 2018/289927, U.S. 2022/096796, U.S. 2022/110695, U.S. 2022/211442, U.S. 2022/233246, U.S. 2023/337985, U.S. 2024/000515, U.S. 2023/414112, U.S. 2022/369934, U.S. 2023/081198, U.S. 2023/097431, U.S. 2023/101030, U.S. 2023/126813, U.S. 2024/099659, each of which are incorporated by reference in their entirety herein.

In an embodiment, a clinician can use the FOSS tracking system 140 to image the medical device 110 and determine the positional information of the medical device distal tip 112 relative to the desired vascular pathway. In an embodiment, a steering magnet 130 can be placed on an exterior of the patient 90 to guide a distal tip 112 of the medical device 110 through the desired vascular pathway.

In an embodiment, the steering magnet 130 is coupled to the FOSS tracking system 140 by way of a FOSS enabled tether 132. The FOSS enabled tether includes a second integrated optical fiber 142B, as described herein, and configured to determine a shape of the tether 132 in three-dimensional space as well as the position of the steering magnet 130 coupled to a distal tip of the tether 132. The console 150 can then track the positional information of the steering magnet 130 relative to the distal tip 112 of the medical device 110 and can display both the medical device distal tip 112 and the steering magnet 130 on the same image, e.g., superimposed on the patient anatomy. Advantageously, this allows the clinician to observe only a single display to observe the medical device 110, the steering magnet 130, and/or the desired vascular pathway and patient anatomy. This mitigates the clinician from having to divert their attention between the image of the medical device 110 and the steering magnet disposed on the patient 90.

In an embodiment, the console 150, including one or both of the FOSS tracking logic 156 and the steering magnet logic 158 can be configured to determine when the medical device distal tip 112 is disposed proximate the location of the steering magnet 130 which is positioned adjacent a target vascular junction. The console 150 can then activate the steering magnet 130, as described herein, as the medical device distal tip 112 approaches the vascular junction (e.g., FIG. 1B). Once the medical device distal tip 112 is advanced past the target location (e.g., FIG. 1C), the console 150 can be configured to deactivate the steering magnet 130. The system 100 can automatically activate the steering magnet 130 when the medical device distal tip 112 is within range of the steering magnet 130 positioned adjacent the target location/vascular junction. The system 100 can activate the steering magnet 130 to modify a direction of the medical device distal tip 112 as the medical device distal tip 112 approaches the target vascular junction, and can automatically deactivate the steering magnet 130 once the medical device distal tip 112 has advanced past the target vascular junction.

Advantageously, the system 100 mitigates human error by automatically activating/deactivating the steering magnet 130 at the appropriate time. The system 100 reduces clinicians work load during complex procedures. The system 100 mitigates unnecessary exposure of electromagnetic radiation to the patient 90 which may affect the patient directly or can affect other nearby systems or medical devices that can be internal or external to the patient 90.

Advantageously, by automatically deactivating the steering magnet 130 once the medical device distal tip 112 has been directed towards the desired vascular pathway, the system 100 prevents the steering magnet 130 from negatively affecting further advancement towards the desired vascular pathway. For example, as shown in FIG. 1B, as the medical device distal tip 112 approaches the target vascular junction, the system 100 can determine when the medical device distal tip 112 is within range of the steering magnet 130 and activate the steering magnet 130 to attract the medical device distal tip 112 towards the desired vascular pathway. Once the medical device distal tip 112 has advanced past the steering magnet 130, the system 100 can automatically deactivate the steering magnet 130 to prevent the steering magnet 130 from further attracting the medical device distal tip 112 which may prevent further distal advancement, or negatively affecting the position of the medical device distal tip 112, e.g., bending the medical device distal tip 112 to a J-shape back towards the steering magnet 130.

In an embodiment, one or both of the medical device distal tip 112 and the steering magnet 130 can further include a radiopaque marker to allow the medical device distal tip 112 and the steering magnet 130 to be observed under fluoroscopy. Advantageously, this allows the clinician to observe only a single display to observe both the medical device 110 and the steering magnet 130, optionally superimposed on an image of the patient and/or the desired vascular pathway. The prevents the clinician from having to divert their attention between the fluoroscopic image of the medical device 110 and the steering magnet 130 disposed on the patient 90.

### Magnetic Tracking System

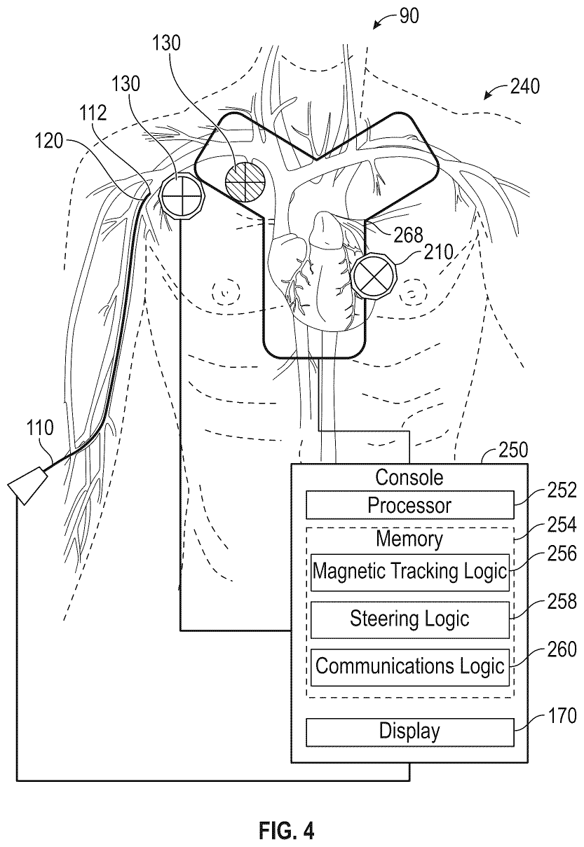

In an embodiment, as shown in FIG. 4, a magnetic tracking system 240 can be used to track the location of the medical device 110 within the body. In an embodiment the magnetic tracking system 240 includes a sensor 268 disposed on an external surface of the patient 90. Optionally, the sensor 268 is disposed adjacent a target location on the external surface of the patient 90 such as the sternum, for example. However, other target locations are also contemplated. The sensor 268 is configured to detect a magnetic field strength emitted by the medical device magnetic element 120.

In an embodiment, a console 250 includes one or more processors 252, non-transitory memory or data storage 254, and one or more logic engines, for example a magnetic tracking logic 256, a steering magnet logic 258, and a communications logic 260. In an embodiment, the console 250, coupled to the sensor 268, can receive magnetic field strength information and determine positional information for the medical device 110, distal tip 112, or combinations thereof. Further details and embodiments of multi-modal imaging and tracking systems can be found, for example, in the following U.S. patents and published patent applications: U.S. Ser. Nos. 8,388,541, 8,971,994, 9,492,097, 9,636,031, 10,238,418, 10,966,630, 11,027,101, US 2018/0116551, US 2018/0304043, US 2019/0069877, US 2019/0099108, US 2020/0054858, US 2020/0237255, and US 2020/0345983, each of which is incorporated by reference in its entirety into this application.

In an embodiment, the magnetic tracking system 240 further includes a steering magnet 130 configured to guide the medical device 110, distal tip 112, magnetic element 120, or combinations thereof, as described herein. In an embodiment, the steering magnet 130 can be a separate device from that of the sensor 268 and can be positioned on the external surface of the patient 90, as described herein. In an embodiment, the steering magnet 130 can be tethered to one or both of the sensor 268 and the console 250. In an embodiment, the steering magnet 130 can be tethered to one or both of the sensor 268 and the console 250 by a FOSS enabled tether 132 as described herein, to track a location of the steering magnet 130 relative to the medical device 110, distal tip 112, sensor 268, combinations thereof, or the like. In an embodiment, the steering magnet 130 can be an electromagnet and can be communicatively coupled with the magnetic tracking system 240. In an embodiment, the steering magnet 130 can be a separate standalone device from one or both of the magnetic based tracking system and the sensor 268 and communicatively coupled therewith by either wired or wireless communication.

In an embodiment, as shown in FIG. 4, the steering magnet 130 can be formed integrally with the sensor 268 and disposed on a bottom surface thereof such that when the sensor 268 is aligned with the sternum of the patient, the steering magnet 130 is aligned with a predetermined position on the external surface of the patient 90. In an embodiment, the console 250 is configured to modify one or both of the magnetic steering magnet 130 and the sensor 268 between an “on” or “activated” state and an “off” or “deactivated” state. In an embodiment, the console 250 is configured to switch the steering magnet 130 and the sensor 268 alternately between the “on” and “off” state such that when the sensor 268 is activated to detect positional information about the medical device 110, the electromagnet of the steering magnet 130 is deactivated so as to mitigate interference with the sensor 268. Similarly, when the steering magnet 130 is activated to steer the medical device 110, the sensor 268 can be deactivated.

In an embodiment, the console 250 can switch between activating the sensor 268/deactivating the steering magnet 130 and activating the steering magnet 130/deactivating the sensor 268 in response to an input from the clinician. In an embodiment, the console 250 can switch between activating the sensor 268/deactivating the steering magnet 130 and activating the steering magnet 130/deactivating the sensor 268 at predetermined time intervals. The predetermined time intervals can be the same length of time or can be different lengths of time from each other. Exemplary time intervals can range between 0.01 Hz and 100 Hz. In an embodiment, activating the sensor 268 and deactivating the steering magnet 130 can be for a longer time interval than deactivating the sensor 268 and activating the steering magnet 130. In an embodiment, activating the sensor 268 and deactivating the steering magnet 130 can be for a shorter time interval than deactivating the sensor 268 and activating the steering magnet 130. These and other combinations of activation/deactivation of the sensor 268/steering magnet 130 are also contemplated.

Advantageously, the console 250 can switch between activating the sensor 268/deactivating the steering magnet 130 and activating the steering magnet 130/deactivating the sensor 268 to prevent the steering magnet 130 from interfering with the sensor 268. Advantageously, the console 250 can switch between activating the sensor 268 and activating the steering magnet 130 at a sufficient rate to provide both tracking and steering at substantially the same time to the perception of the clinician, while mitigating interference between steering magnet 130 and the sensor 268. Worded differently, the console 250 can switch between activating the sensor 268 and activating the steering magnet 130 at a rate where a length of time between a first activation of the sensor 268 and a second activation of the sensor 268 is so short that little movement has occurred during the intervening deactivation period.

In an embodiment the magnetic tracking system 240 including the sensor 268 can be placed on the sternum of the patient and can be configured to detect the presence of an electro-magnetic impulse. The electro-magnetic impulse can be provided by the medical device magnetic element 120, or from another medical device 210 disposed within the patient, such as for example, a pacemaker 210, or the like.

The console 250 can be configured to receive information from the sensor 268 about the electromagnetic signal and determine the positional and identity information about the medical device 110 and/or the alternate medical device 210. For example, the sensor 268 can detect time, frequency, and/or amplitude information to determine the positional and/or identity information of the medical device 110 and/or the alternate medical device 210. For example, the console 250 can determine the presence of a pacemaker 210 from the pattern of electrical or electro-magnetic impulses in the time domain and differentiate these signals from that of the medical device magnetic element 120.

Advantageously, the magnetic tracking system 240 can provide a visual, audible and/or tactile alert to the clinician indicating the presence of the alternate medical device 210 and how this might affect the sensor 268 from detecting the medical device 110. Further, the console 250 can automatically deactivate one or more of the sensor 268, steering magnet 130, and medical device magnetic element 120, or combinations thereof, to prevent the magnetic tracking system 240 and/or medical device 110 from interfering with or disrupting the alternate medical device 210.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A tracking and steering system comprising:
a medical device including a magnetic element coupled with a distal portion of the medical device;
a sensor placed on an external surface of a patient and configured to detect a magnetic field strength of the magnetic element of the medical device;
a steering magnet disposed on the external surface of the patient and including an electromagnet configured to attract the magnetic element of the medical device;
a console including memory and one or more processors configured to:
receive magnetic field strength information from the sensor and determine positional information for a distal tip of the medical device; and
simultaneously activate the sensor and deactivate the steering magnet or simultaneously activate the steering magnet and deactivate the sensor; and
a display configured to receive positional information from the console and display an image of the patient and display an icon representing a location of the medical device relative to the patient.

2. The tracking and steering system according to claim 1, wherein the medical device includes one or more of a catheter, guidewire, or stylet.

3. The tracking and steering system according to claim 1, wherein the magnetic element of the medical device is a permanent magnet or a magnetically responsive material.

4. The tracking and steering system according to claim 1, wherein the console is configured to receive an input from a clinician to switch between simultaneously activating the sensor and deactivating the steering magnet, and simultaneously activating the steering magnet and deactivating the steering magnet.

5. The tracking and steering system according to claim 1, wherein the console is further configured to switch between simultaneously activating the sensor and deactivating the steering magnet, and simultaneously activating the steering magnet and deactivating the sensor at between 5 Hz and 50 Hz.

6. The tracking and steering system according to claim 1, wherein the positional information includes location, orientation, or shape of the medical device.

7. The tracking and steering system according to claim 1, wherein the steering magnet is formed integrally with the sensor to align the steering magnet with a predefined location when the sensor is disposed over a sternum of the patient.

8. The tracking and steering system according to claim 1, wherein the steering magnet is formed as a separate standalone device from the sensor and is communicatively coupled with one or both of the sensor and the console by wired or wireless communication.

9. The tracking and steering system according to claim 1, wherein the steering magnet is coupled to a garment that is worn by the patient and aligns the steering magnet with a predetermined location on an exterior of the patient.

10. The tracking and steering system according to claim 1, wherein one or both of the sensor and the medical device includes an electrode configured to detect an electrical impulse, and wherein the console is configured to determine a presence of a pacemaker, and in response to the console determining the presence of the pacemaker, the console prevents the steering magnet from being activated.

11. The tracking and steering system according to claim 10, wherein the console provides one or more of a visual, audible, and tactile alert to a clinician to indicate the steering magnet is deactivated due to the presence of the pacemaker being detected.

12. The tracking and steering system according to claim 1, wherein a polarity of the electromagnet is reversed to change the steering magnet between attracting and repelling the medical device magnetic element.

13. A tracking and steering system comprising:
a medical device comprising:
a first magnetic element coupled with a distal portion thereof; and
a first integrated optical-fiber having a plurality of fiber Bragg grating (“FBG”) sensors along a portion thereof;
a steering magnet disposed on an external surface of a patient comprising:
a second magnetic element; and
a tether extending from the steering magnet and including a second integrated optical-fiber having a plurality of FBG sensors along a portion thereof;
a console including memory and one or more processors configured to:
convert FBG sensor-reflected optical signals from the first integrated optical-fiber in the medical device and the second integrated optical-fiber in the tether into plottable data by way of a plurality of optical signal-converter algorithms to determine position information for a distal tip of the medical device and the steering magnet; and
a display configured to provide an image of the patient, a first icon indicating a position of the medical device, and a second icon indicating a position of the steering magnet relative to each other.

14. The tracking and steering system according to claim 13, wherein the medical device includes one of a catheter, guidewire, or stylet.

15. The tracking and steering system according to claim 13, wherein the first magnetic element of the medical device is one of a permanent magnet or a magnetically responsive material, and wherein the second magnetic element of the steering magnet is one of a permanent magnet or an electromagnet.

16. The tracking and steering system according to claim 13, wherein a positional information of the distal tip of the medical device and the steering magnet includes location, orientation, or shape.

17. The tracking and steering system according to claim 13, wherein the tether is coupled to the console.

18. The tracking and steering system according to claim 13, wherein the console further includes one or more logic engines configured to determine a position of the distal portion of the medical device relative to a position of the steering magnet and activate the steering magnet when the distal portion of the medical device approaches a target location and deactivate the steering magnet when the distal portion of the medical device is advanced past the target location.

19. A tracking and steering system comprising:
a medical device including a magnetic element coupled with a distal portion of the medical device;
a sensor placed on an external surface of a patient and configured to detect a magnetic field strength of the magnetic element of the medical device;
a steering magnet disposed on the external surface of the patient and including an electromagnet configured to attract the magnetic element of the medical device, the steering magnet including a tether having an integrated optical-fiber having a plurality of fiber Bragg grating (“FBG”) sensors along a portion thereof;
a console including memory and one or more processors configured to:
receive magnetic field strength information from the sensor and determine positional information for a distal tip of the medical device;
simultaneously activate the sensor and deactivate the steering magnet or simultaneously activate the steering magnet and deactivate the steering magnet; and
convert FBG sensor-reflected optical signals from the integrated optical-fiber in the tether into plottable data by way of a plurality of optical signal-converter algorithms to determine position information for the steering magnet; and
a display configured to receive positional information from the console and display an image of the patient, display a first icon representing a location of the medical device relative to the patient, and a second icon representing a location of the steering magnet.

20. A method of placing a medical device, comprising:
advancing a distal tip of the medical device intravascularly, the distal tip including a medical device magnetic element;
placing a steering magnet on an external surface of a patient to modify a position of the distal tip disposed intravascularly, the steering magnet including an electromagnetic element;
detecting a magnetic field strength of the medical device magnetic element by way of a sensor placed on the external surface of the patient; and
modifying an activation state of the steering magnet and the sensor, by way of a console, to alternate between simultaneously activating the steering magnet and deactivating the sensor, and simultaneously deactivating the steering magnet and activating the sensor at a rate of between 5 Hz and 50 Hz.
